(19) United States
(12) Patent Application Publication
(10) Pub. No.: US 2021/0390366 A1
(43) Pub. Date: Dec. 16, 2021

(54) Empathic Computing System and Methods for Improved Human Interactions With Digital Content Experiences

(71) Applicant: Arctop LTD, Tel Aviv (IL)

(72) Inventors: Daniel Furman, San Francisco, CA (US); Eitan Kwalwasser, Tel Aviv (IL)

(21) Appl. No.: 17/285,082

(22) PCT Filed: Oct. 25, 2019
(86) PCT No.: PCT/US19/58057

§ 371 Date: Apr. 13, 2021

Related U.S. Application Data
(60) Provisional application No. 62/750,255, filed on Oct. 25, 2018.
(60) Provisional application No. 62/871,435, filed on Jul. 8, 2019.

(51) Int. Cl. G06N 3/00; G06N 3/08

(52) U.S. Cl. CPC G06N 3/008; G06N 3/08

(57) ABSTRACT

The invention(s) described relate generally to synthetic brain models implementing computer operations that are configured to understand human thoughts and feelings and modulate content accordingly, with the aim of providing better, more personalized service (e.g., in the context of entertainment, training, health, security, etc.). The empathic computing system executing the synthetic brain model(s) described brings utility to evaluation of digital content experiences (e.g., involving mixed media formats) provided to users in their daily lives (e.g., with respect to audio content, with respect to visual content, with respect to content of other formats, with respect to connected home applications, with respect to AR/VR device applications, with respect to automotive technology applications, etc.).

### CROSS REFERENCE TO RELATED APPLICATIONS

This application claims the benefit of U.S. Provisional Application Ser. No. 62/750,255 filed 25 Oct. 2018 and U.S. Provisional Application Ser. No. 62/871,435 filed 8 Jul. 2019, which are each incorporated in its entirety herein by this reference.

### BACKGROUND

The present disclosure generally relates to neural signal processing, and specifically to a system and method for interactive content delivery coordinated with rapid decoding of brain activity, using a brain-computer interface.

Brain-computer interface (BCI) systems and methods can be used to interface users seamlessly with their environment and to enhance user experiences in digital worlds. Such BCI systems can be used to generate neural signal data from users as they interact with digital content and/or have other experiences in their daily lives, in order to contribute to feedback mechanisms for providing users with customized or improved content. In relation to delivery of customized content, current systems are unable to rapidly decode neurological activity of a user and to coordinate decoding with provision of digital content tailored to users. Current systems are further unable to evaluate content and unable to predict how users will respond to that content with suitable levels of resolution.

### SUMMARY

The invention(s) described relate generally to synthetic brain models implementing computer operations that are configured to understand human thoughts and feelings and modulate content accordingly, with the aim of providing better, more personalized service (e.g., in the context of entertainment, training, health, security, etc.). The empathic computing system executing the synthetic brain model(s) described brings utility to evaluation of digital content experiences (e.g., involving mixed media formats) provided to users in their daily lives (e.g., with respect to audio content, with respect to visual content, with respect to content of other formats, with respect to connected home applications, with respect to AR/VR device applications, with respect to automotive technology applications, etc.). In embodiments, the digital content experience can include one or more of: an audio listening experience, a video watching experience, an image viewing experience, a text reading experience, a shopping experience, and a video gameplay experience provided by way of a digital content file.

The invention(s) described also relate to content creation, with respect to evaluation of predicted responses of users (or demographics of users) to created content and/or with respect to generation or modulation of created content based on predicted responses of users (or demographics of users) to content.

The invention(s) described also relate to the development of a synthetic brain in software, where human neural signals and/or other physiological signals analyzed by one system (a “first subsystem”) are processed with environmental signals and features of provided digital content analyzed by a second system (a “second subsystem”) to train a synthetic brain model, which collectively pools insights and develops a matrix of human experiential states related to responses to different experiences. Content features (e.g., data parameters or other aspects of electronic content) associated with the second subsystem are fed into a network that uses computer vision and speech recognition techniques, while neural signal data captured from a brain-computer interface (BCI) associated with the first expert system is processed using unique software developed specifically for this purpose. Combined insights from both subsystems enable the synthetic brain model, with refinement, to learn statistical relationships and make predictions on future data from a single stream only (e.g., stream related to features of content only, or stream related to features from brain signals only). As such, the subsystems include architecture for implementation of methods that can be used to generate predictions (e.g., of user responses, of content effectiveness, of portion of content being interacted with by a user, etc.) using a single data stream.

In more detail, with increased amounts of training data and model refinement, the system architecture (e.g., a matrix defined by the system architecture) refines models of human perception, emotion, reactions, decisions and other human experiential states that may then be used to forecast human-like experiences or other behavioral response from environmental input data (e.g., digital content data) only. Such a system, capable of emulating human experiences, is a valuable tool for improving artificial intelligence programs and autonomous systems that interact with humans, as well as editing and improving digital content presented to humans, for example video content (e.g., film, TV, games), and audio content (e.g., music, sound effects, virtual assistant voice features, etc.), whose impact can be enhanced by creators that have an informed view into the emotional effect certain creative decisions have. The system can further generate models for emulating human experiences or responses across different demographics or other categories of individuals.

In embodiments, the system can also receive neural signal data from one or more subjects as the subject(s) interact(s) with content, and the system can output predicted portions of the content based on processing of the neural signal data. As such, the system can be trained to predict what portions of digital content users are consuming based upon analysis of neural signal data alone.

In embodiments, the system can also be used to identify or predict differential responses to (e.g., in terms of empathic responses, in terms of behavioral responses, etc.) to the same content (e.g., an audio clip, a video clip, other unevaluated content) based on implementation of synthetic brain models trained with data from a population of users.

In embodiments, the system can be configured to identify unexpected clusters of subjects or other markets for targeting content, based on similar responses of such subjects to provided content. As such, the system can be used as a diagnostic tool to identify new markets or new demographics not previously characterizable by other methods.

In embodiments, the system can be configured to predict user responses to other interactive content (e.g., features of video games), in relation to designable features of gameplay for such content.

In relation to virtual assistants operating in a digital environment, embodiments of the system can be used to test or generate features of virtual assistant interactions, using a synthetic brain model. As such, outputs of the system can be used as an antidote to virtual assistant interactions that give humans the uncomfortable feeling that they are interacting with an entity that suffers from alexithymia—a personality construct characterized by the subclinical inability to identify and describe emotions in the self, and a dysfunction in emotional awareness, social attachment, and interpersonal relating. By communicating information about the human user's personal perspective in the form of various data that characterize experiential states, machines can operate much more intuitively and sensitively, for instance, by providing more natural user-system interactions.

### BRIEF DESCRIPTION OF THE DRAWINGS

FIG. 1A depicts a schematic of a system environment for synthetic brain development and implementation, in accordance with one or more embodiments.

FIG. 1B depicts an embodiment of an application environment for synthetic brain model implementation.

FIG. 2 depicts a flowchart of a method for synthetic brain development and implementation, in accordance with one or more embodiments.

FIG. 3A depicts an embodiment of architecture for classification of externally-derived features and internally-derived features, outputs of which are used to develop, train, and/or otherwise refine synthetic brain models for generating predicted user responses to unevaluated content.

FIG. 3B depicts an embodiment of implementation of the synthetic brain model trained as described in relation to FIG. 3A.

FIG. 3C depicts an embodiment of real-time analysis of content data, using the synthetic brain model trained as described in relation to FIG. 3A

FIG. 4A (top) depicts a circumplex model (i.e. PAD model) and FIG. 4A (bottom) depicts a second model that maps human emotion to a 27-dimensional space connected at times by smooth gradients, that can be used to develop synthetic brain models.

FIG. 4B depicts example graphs depicting hierarchical clustering of subjects by features.

FIG. 5A depicts a series of charts correlating example outputs of a synthetic brain model to self-reported responses to consuming of provided content by users.

FIG. 5B depicts example model outputs generating predictions of user responses, according to operations described above, where response predictions were generated for different demographics (e.g., pop fans, genders, surfers, ages, yoga-ists, locations, etc.).

FIG. 6 depicts example output metrics related to empathic responses (e.g., relaxation, anxiety, enjoyment, boredom, etc.) and other responses across each segment of and through an entire duration of evaluated song, for different demographics (e.g., male, female, American, Israeli, other nationality, etc.).

FIG. 7 depicts output metrics produced by a synthetic brain model, where outputs are related to empathic responses (e.g., relaxation, anxiety, enjoyment, boredom, engagement, difficulty, etc.) in relation to different game play factors of a video game experience

FIG. 8 depicts examples graphics corresponding to outputs of synthetic brain models used to process input neural signal data, in order to produce output predictions of portions of digital content being consumed (based on evaluation of neural signal data alone).

### DETAILED DESCRIPTION

### 1. System Environment

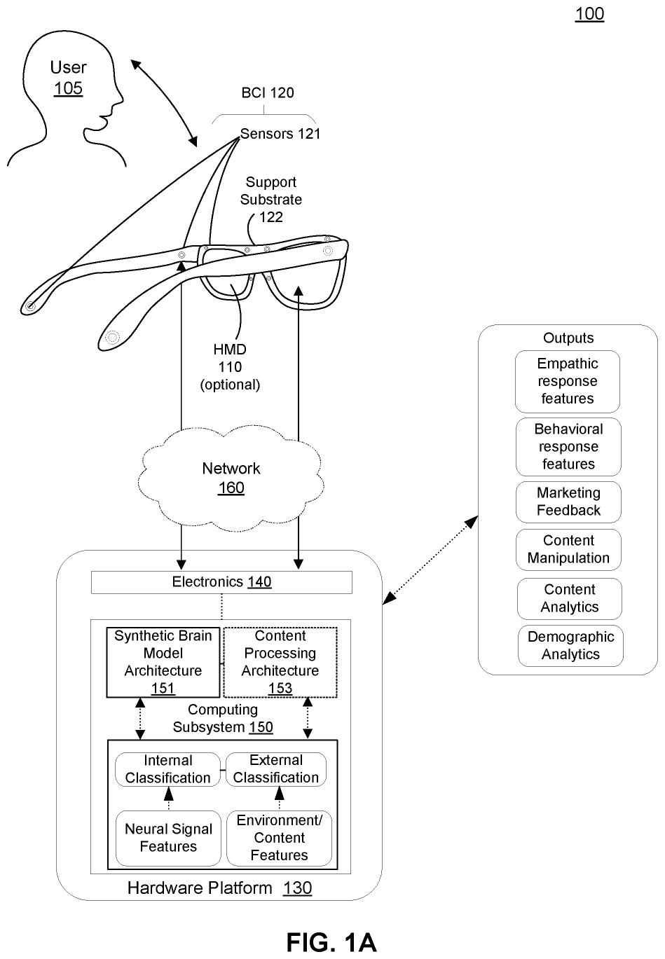

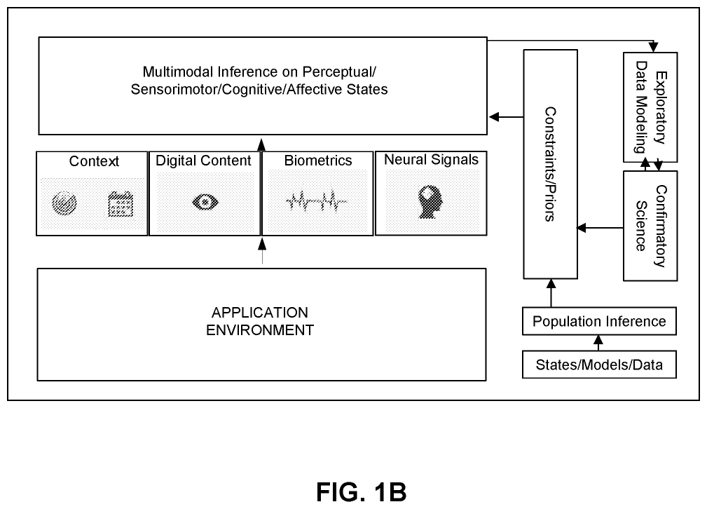

FIG. 1A depicts a system environment of a system 100 synthetic brain development and implementation, in accordance with one or more embodiments. The system 100 shown in FIG. 1A includes a brain-computer interface (BCI) 120 including an array of sensors from which a neural signal dataset from a user 105 is generated, as the user interacts with digital content. The BCI 120 can be coupled to or otherwise cooperate with a head-mounted display (HMD) 110, and the digital content can be provided through the HMD and/or through another device (e.g., a device capable of rendering video and/or outputting audio signals to a user). The system 100 also includes a hardware platform 130 configured to couple with the HMD 110 and/or the BCI 120, where the hardware platform 130 includes an electronics subsystem 140 for receiving and conditioning outputs of the BCI 120, as well as architecture for classifying features of digital content provided to users, environment signals, neural signals, and/or other physiological signals, and developing, training, and implementing synthetic brain model outputs for performance of subsequent actions.

The embodiments of the system 100 function to receive and process digital content features, neural signals, environmental signals, and/or other data to develop and train synthetic brain models capable of processing reduced data streams (in type and/or content) and generating actionable outputs. In embodiments, the system 100 can function to promote content creation, with respect to evaluation of predicted responses of users (or demographics of users) to created content and/or with respect to generation or modulation of created content based on predicted responses of users (or demographics of users) to content. With training of synthetic brain models, embodiments of the system 100 are capable of emulating human experiences, as a valuable tool for improving artificial intelligence programs and autonomous systems that interact with humans, as well as editing and improving digital content presented to humans, for example video content (e.g., film, TV, games), and audio content (e.g., music, sound effects, virtual assistant voice features, etc.), whose impact can be enhanced by creators that have an informed view into the emotional effect certain creative decisions have. Embodiments of the system 100 can further generate models for emulating human experiences or responses across different demographics or other categories of individuals.

In embodiments, the system 100 can additionally or alternatively receive neural signal data from one or more subjects as the subject(s) interact(s) with content, and the system can output predicted portions of the content based on processing of the neural signal data. As such, the system can be trained to predict what portions of digital content users are consuming based upon analysis of neural signal data alone. In embodiments, the system 100 can also be configured to identify unexpected clusters of subjects or other markets for targeting content, based on similar responses of such subjects to provided content. As such, the system can be used as a diagnostic tool to identify new markets or new demographics not previously characterizable by other methods. The system 100 can be configured to implement or execute embodiments of the methods described below, or can additionally or alternatively be configured to execute other methods related to application of synthetic brains for improving content provided to users.

### 1.1 System—BCI and HMD

As shown in FIG. 1A, the BCI 120 includes a set of sensors 121 configured to detect neurological activity from the brain of the user, during use. In one embodiment, the set of sensors 121 include electrodes for electrical surface signal (e.g., electroencephalogram (EEG) signal, electromagnetic field signal, electrocorticography (ECoG) signal, etc.) generation, where the set of sensors 121 can include one or more of electrolyte-treated porous materials, polymer materials, fabric materials, or other materials that can form an electrical interface with a head region of a user. In alternative embodiments, the set of sensors 121 can include sensors operable for one or more of: magnetoencephalography (MEG), positron emission tomography (PET), functional magnetic resonance imaging (fMRI), single neuron signal sensing (e.g., using neurotrophic electrodes, using multi-unit arrays), and other neurosensing modalities. In still alternative embodiments, the set of sensors 121 can include sensors operable for optical neurosensing modalities including one or more of: diffuse optical tomography (DOT), near-infrared spectroscopy (fNIRS), functional time-domain near-infrared spectroscopy (TD-fNIRS), diffuse correlation spectroscopy (DCS), speckle contrast optical tomography (SCOT), time-domain interferometric near-infrared spectroscopy (TD-iNIRS), hyperspectral imaging, polarization-sensitive speckle tomography (PSST), spectral decorrelation, and other imaging modalities.

As shown in FIG. 1A, the sensors 121 of the BCI 120 can be coupled to a support substrate 122, where the support substrate 122 can include portions configured to arch over a frontal and/or pre-frontal portion of the head of the user during use, as well as temporal portions, parietal portions, and maxillofacial regions of the user's head. In embodiments, the support substrate 122 can form one or more of: frames, temple pieces, and nose bridge of eyewear of another device (e.g., the HMD 110 described in more detail below), such that the user is provided with display and sensing functionality in a compact form factor. As shown in FIG. 1A, the sensors 121 of the BCI 120 are coupled inward facing portions of the temple pieces, frame, and nose bridge of the support substrate 122 to interface with appropriate portions of the user's head and/or face during use. As such, BCI 120 can share computing components, power management components, and/or other electronics with other head mounted objects (e.g., the HMD 110 described in more detail below) in a configuration as a single apparatus. The system can be integrated with head mounted objects that are worn primarily for fashion or functional purposes, such as baseball hats, and can be configured to provide real-time outputs about the wearer's brain both locally, i.e. on the apparatus itself, or remotely, i.e. in a cloud-connected application.

In some embodiments, the system 100 can also include devices for providing digital content (e.g., audio content, visual content, haptic content, consumer experiences, olfaction, etc.) to users. For instance, the system 100 can additionally or alternatively include an HMD 110 configured to be worn by a user and to deliver digital content generated by the architecture of the hardware platform 130 to the user. The HMD 110 includes a display for rendering electronic content to a user. As described in relation to the methods below, content rendered by the display of the HMD 110 can include digital content and/or virtual environments 109 within a field of view associated with the display. The digital objects 107 and/or virtual environments 109 have modulatable features that can be used to prompt interactions with a user, as described below. The HMD 110 can additionally include one or more of: power management-associated devices (e.g., charging units, batteries, wired power interfaces, wireless power interfaces, etc.), fasteners that fasten wearable components to a user in a robust manner that allows the user to move about in his/her daily life, and any other suitable components. The HMD 110 can also include interfaces with other computing devices, such as a mobile computing device (e.g., tablet, smartphone, smartwatch, etc.) that can receive inputs that contribute to control of content delivered through the HMD 110, and/or deliver outputs associated with use of the HMD 110 by the user. As indicated above, however, the HMD 110 can be replaced or supplemented with any other suitable device(s) operable to render or output content to users.

Furthermore, as shown in FIG. 1A, in embodiments the BCI 120 can be coupled to one or more portions of the HMD 110, such that the user wears a single apparatus having both content provision functions and neurological signal detection and transmission functions. For instance, the sensors 121 of the BCI 120 can be coupled to a support substrate 122 configured to arch over a frontal and/or pre-frontal portion of the head of the user during use, where the sensors 121 of the BCI 120 are coupled to a posterior portion of the support substrate 122 to contact the head of the user during use. In some embodiments, terminal regions of the support substrate 122 are coupled to (e.g., electromechanically coupled to, electrically coupled to, mechanically coupled to) to bilateral portions of housing portions of the HMD 110. As such, the HMD 110 and the BCI 120 can share computing components, power management components, and/or other electronics.

However, in still alternative embodiments, the components of the BCI 120 can be coupled to the HMD 110 in another manner. In still alternative embodiments, the BCI 120 can be physically distinct from the HMD 110, such that the BCI 120 and the HMD 110 are not configured as a single apparatus.

### 1.2 System—Hardware Platform

As shown in FIG. 1A, the hardware platform 130 includes a computing subsystem 150 in communication with an electronics subsystem 140, where the electronics subsystem 140 includes components for facilitating transmission of data over network 160 (described in more detail below), power management, pre-processing of data, and/or conditioning of signals communicated between components of the system 100. Furthermore, the computing subsystem can include a nontransitory computer-readable storage medium containing computer program code for operating in different modes associated with content provision, acquisition of data (e.g., related to neural signals, related to factors of the user's environment, etc.), and/or processing of data with model architecture (e.g., related to implementation of classification operations, related to training of synthetic brain models, related to processing of inputs and generation of outputs by synthetic brain models, etc.).

The computing subsystem 150 can thus include synthetic brain model architecture 151 that allows the system 100 to process outputs of classification operations (e.g., governed by internal classification architecture that processes neural signal features from the BCI 120 and/or external classification architecture that processes features of the environment and/or provided content) in order to produce a set of outputs. In examples, outputs can be associated with predicted empathic responses by users to provided content, predicted behavioral responses by users to provided content, marketing feedback (associated with aspects of the digital content provided), outputs associated with content generation and manipulation, analytics associated with provided content, analytics associated with demographics for which the content is being targeted, and/or other outputs, as described in more detail below.

In relation to content generation and manipulation, the computing subsystem can also include content processing architecture 153 that includes subcomponents associated with content provision (e.g., through various display devices described above), content generation (e.g., in relation to generation of content in video formats, image formats, audio formats, haptic formats, etc.), and/or content manipulation. As such, the system 100 can be configured to process outputs of the iterations of the synthetic brain model of the computing subsystem 150, and to use outputted predicted user responses to provide analytics and/or generate or manipulate previously unevaluated content to better suit users or target demographics.

The computing subsystem 150 can thus include computing subsystems implemented in hardware modules and/or software modules associated with one or more of: personal computing devices, remote servers, portable computing devices, cloud-based computing systems, and/or any other suitable computing systems. Such computing subsystems can cooperate and execute or generate computer program products comprising non-transitory computer-readable storage mediums containing computer code for executing embodiments, variations, and examples of the methods described below. As such, portions of the computing subsystem 150 can include architecture for implementing embodiments, variations, and examples of the methods described below, where the architecture contains computer program stored in a non-transitory medium.

### 1.3 System—Communications

As shown in FIG. 1A, the components of the system 100 can be configured to communicate with the through network 160, which can include any combination of local area and/or wide area networks, using both wired and/or wireless communication systems. In one embodiment, the computing subsystem 150 and/or other devices of the system (e.g., HMD 110, BCI 120) use standard communications technologies and/or protocols. For example, the network 160 includes communication links using technologies such as Ethernet, IEEE 802.11, worldwide interoperability for microwave access (WiMAX), 3G, 4G, 5G, code division multiple access (CDMA), global system for mobile communications (GSM), digital subscriber line (DSL), etc. Examples of networking protocols used for systems communication include transmission control protocol/Internet protocol (TCP/IP), hypertext transport protocol (HTTP), WebSocket (WS), and file transfer protocol (FTP). In some embodiments, all or some of the communication links of components of the system 100 may be encrypted using the secure extension of said protocol such as hypertext transfer protocol over secure sockets layer (SSL), WebSocket secure (WSS), secure file transfer program (SFTP) or any other suitable technique or techniques.

### 1.4 System—Other Sensors and Elements

Devices of the system 100 can include additional sensor components for detecting aspects of user states, detecting contextual information (e.g., from a real-world environment of the user), and/or detecting aspects of interactions with electronic content generated by the computing subsystem 150 and transmitted through the HMD 110 and/or other devices. Subsystems and/or sensors of can be coupled to, integrated with, or otherwise associated with the HMD 110 and/or BCI 120 worn by the user during interaction with provided content. Subsystems and/or sensors can additionally or alternatively be coupled to, integrated with, or otherwise associated with devices distinct from the BCI 120, HMD 110, and/or other devices and communicate with the computing subsystem 150 during interactions between the user and provided digital content experiences.

Additional sensors can include audio sensors (e.g., directional microphones, omnidirectional microphones, etc.) to process captured audio associated with a user's interactions with the electronic content and/or environments surrounding the user. Sensors can additionally or alternatively include optical sensors (e.g., integrated with cameras) to process captured optically-derived information (associated any portion of an electromagnetic spectrum) associated with a user's interactions with the electronic content and/or environments surrounding the user (e.g., with respect to eye tracking, with respect to facial feature or expression detection). Sensors can additionally or alternatively include motion sensors (e.g., inertial measurement units, accelerometers, gyroscopes, etc.) to process captured motion data associated with a user's interactions with the electronic content and/or environments surrounding the user. Sensors can additionally or alternatively include biometric monitoring sensors including one or more of: skin conductance/galvanic skin response (GSR) sensors, sensors for detecting cardiovascular parameters (e.g., radar-based sensors, photoplethysmography sensors, electrocardiogram sensors, sphygmomanometers, etc.), sensors for detecting respiratory parameters (e.g., plethysmography sensors, audio sensors, etc.), body temperature sensors, and/or any other suitable biometric sensors. As such, additional sensor signals can be used by the hardware platform 130 for extraction of non-brain activity states (e.g., auxiliary biometric signals, auxiliary data, contextual data, etc.) that are relevant to determining user states. For instance, environmental factors (e.g., an analysis of environmental threats) and/or devices states (e.g., a user's device is wirelessly connected or connected otherwise to a network) can be used as inputs. The system 100 can thus process outputs of the sensors to extract features useful for guiding content modulation in near-real time according to the method(s) described below.

FIG. 1B depicts an embodiment of an application environment for synthetic brain model implementation. During operation of the system and application environment, multimodal data is acquired from one or more users or other entities, and the application environment is used to make high-level inferences which directly modulate application environment and produce outputs of synthetic brain models. The high-level inferences produced by the computing subsystem can contemporaneously generate new models that can be used to reverse engineer efficient physiological markers of given states and experiences. As shown in FIG. 1B, the application environment can facilitate multimodal fusing of internally and externally-derived data, produce outputs of synthetic brain models by multimodal inference, implement exploratory data modeling techniques with confirmatory science under boundary constraints (e.g., based on physiology, based on constraints defined by external factors, etc.), and process population-wide or demographic-specific inferences.

While the system(s) described above preferably implement embodiments, variations, and/or examples of the method(s) described below, the system(s) can additionally or alternatively implement any other suitable method(s).

### 2. Method—Synthetic Brain Development, Refinement, and Implementation

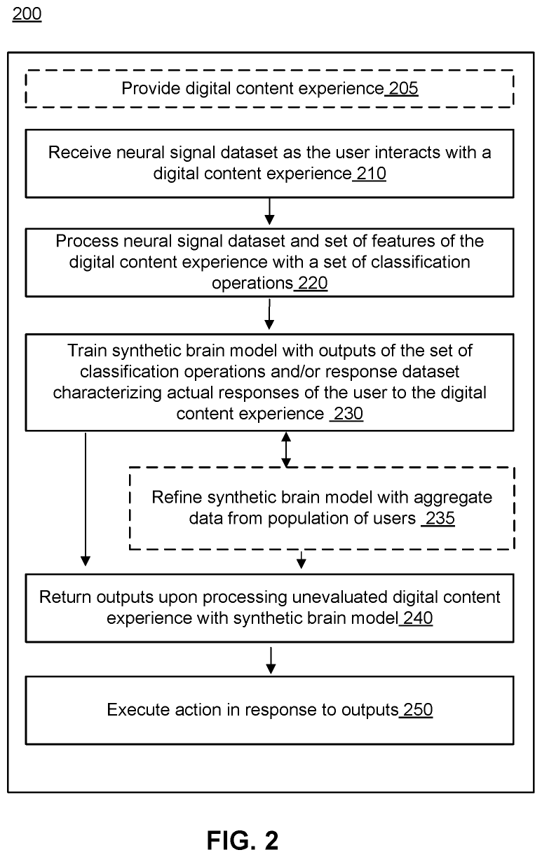

FIG. 2 depicts a flowchart of a method 200 for synthetic brain development and implementation, in accordance with one or more embodiments. As shown in FIG. 2, the hardware platform and associated computing subsystem receives 210 a neural signal dataset from a BCI coupled to the user, as the user interacts with a digital content experience. Then, the computing subsystem processes 220 the neural signal dataset and a set of features of the digital content experience with a set of classification operations. Then, the computing subsystem trains 230 a synthetic brain model with outputs of the set of classification operations and a response dataset characterizing actual responses of the user to the digital content experience, the synthetic brain model comprising architecture for returning outputs associated with predicted user responses to digital content experiences. Then, the computing subsystem returns 240 a set of empathic and behavioral outputs associated with predicted user responses to an unevaluated digital content experience, upon processing the unevaluated digital content experience with the synthetic brain model. The computing subsystem then executes 250 an action in response to the set of empathic and behavioral outputs. In some embodiments, the system (e.g., an embodiment of the system described above) can perform any one or more of: providing 205 a digital content experience to a user and refining 235 the synthetic brain model with an aggregate dataset comprising neural signal data from a population of users, thereby enabling the synthetic brain model to output demographic-related analytics in relation to digital content being evaluated.

The embodiments of the method 200 function to receive and process digital content features, neural signals, environmental signals, and/or other data to develop and train synthetic brain models capable of processing reduced data streams (in type and/or content) and generating actionable outputs. In embodiments, the method 200 can function to promote content creation, with respect to evaluation of predicted responses of users (or demographics of users) to created content and/or with respect to generation or modulation of created content based on predicted responses of users (or demographics of users) to content. With training of synthetic brain models, embodiments of the method 200 are capable of emulating human experiences, as a valuable tool for improving artificial intelligence programs and autonomous systems that interact with humans, as well as editing and improving digital content presented to humans, for example video content (e.g., film, TV, games), and audio content (e.g., music, sound effects, virtual assistant voice features, etc.), whose impact can be enhanced by creators that have an informed view into the emotional effect certain creative decisions have. Embodiments of the method 200 can further generate models for emulating human experiences or responses across different demographics or other categories of individuals. Emulated human experiences produced by the models can then be used to strategically provide content to receptive demographics, thereby reducing wasted efforts in targeting the content to less receptive audiences. Emulated human experiences also enable autonomous content creation loops (machine-driven) that enable media and entertainment applications to iterate rapidly in generating new content without the constraint of needing live humans to serve as test audiences. The scale of testing (e.g., number of people emulated) and rate (e.g., how fast results are provided) enabled by such a system confers considerable advantage to media and entertainment creators over traditional development processes.

In embodiments, the method 200 can additionally or alternatively receive neural signal data from one or more subjects as the subject(s) interact(s) with content, and the system can output predicted portions of the content based on processing of the neural signal data. As such, the system can be trained to predict what portions of digital content users are consuming based upon analysis of neural signal data alone. In embodiments, the system 100 can also be configured to identify unexpected clusters of subjects or other markets for targeting content, based on similar responses of such subjects to provided content. As such, the method 200 can be used as a diagnostic tool to identify new markets or new demographics not previously characterizable by other methods. The method 200 can be implemented or executed by embodiments of the systems described above, or can additionally or alternatively executed by other systems or system components having functionality for implementation of synthetic brain models.

### 2.1 Method—Content

In relation to providing 205 a digital content experience to a user, the digital content can include one or more formats including: video file formats (e.g., MP4, 3GP, OGG, WMV, WEBM, FLV, AVI, QuickTime™, stereoscopic formats, etc.), audio file formats (e.g., WAV, AIFF, AU, PCM, FLAC, MPEG, WMA, OPUS, MP3, etc.), image file formats (e.g., JPEG, TIFF, GIF, EXIF, BMP, PNG, HDR, vector formats, stereoscopic formats, etc.), haptic file formats (e.g., AHAP), video game formats (e.g., with respect to PC platforms, home console platforms, handheld platforms, arcade platforms, web browser platforms, mobile device platforms, virtual reality platforms, augmented reality platforms, blockchain platforms, etc.), and any other suitable formats.

The digital content can be associated with categories of experiences including one or more of: video watching (e.g., in association with an advertisement, in association with a full-length movie, in association with a short movie, in association with a TV show episode, in association with a movie clip, in association with augmented reality experiences, in association with virtual reality experiences, etc.), audio listening (e.g., in association with an advertisement, in association with a song, in association with a composition, in association with a playlist, in association with an audio clip, in association with a virtual assistant experiences, etc.), shopping experiences (e.g., in association with an advertisement, in association with shopping online, in association with shopping through an application, in association with shopping in another retail environment, etc.), text interaction experiences (e.g., with respect to reading digital written content), gameplay experiences (e.g., in association with a video game, in association with a board game, in association with an “escape the room”-style experience, in association with a mobile device game, etc.), in association with a learning experience (e.g., in a teaching environment, in a virtual environment, in relation to learning software, etc.), and any other suitable experience.

Features of the digital content can be associated with anticipated empathic responses, for instance, in relation to emotion-affecting content. Features of the digital content can additionally or alternatively be associated with anticipated behavioral responses (e.g., in relation to selection of content for purchase, in relation to performance of actions related to content, in relation to selection of content for a playlist or library, in relation to engagement with content, etc.).

As such, features of video content can include subject matter features (e.g., subject matter having a degree of conflict, subject matter having a degree of conflict resolution, subject matter having a degree of love-associated content, subject matter having a degree of adventure-associated content, subject matter having a degree of positive emotionality, subject matter having a degree of negative emotionality, subject matter targeted to adults, subject matter targeted to children, subject matter targeted to other age groups, subject matter targeted to different ethnicities, subject matter targeted to different nationalities, subject matter targeted to different cultures, historical subject matter, present time subject matter, futuristic subject matter, subject matter having a certain degree of realism, subject matter including celebrities, subject matter including non-celebrities, etc.), degree of live action content, degree of animated content, level of special effects, and/or other subject matter features, where subject matter aspects can be assessed qualitatively (e.g., categorically) and/or quantitatively (e.g., with scoring). Features of video content can additionally or alternatively be associated with one or more of: duration (of entire video, of scenes, of other subportions), frame rate, format (e.g., wide angle, stereoscopic, etc.), resolution (e.g., 4K, 8K, etc.), gauge (e.g., super 8, 16 mm, 35 mm, 65 mm, etc.), distortion features, and any other suitable technical feature.

Similarly, features of image content can include subject matter features (e.g., subject matter having a degree of conflict, subject matter having a degree of conflict resolution, subject matter having a degree of love-associated content, subject matter having a degree of adventure-associated content, subject matter having a degree of positive emotionality, subject matter having a degree of negative emotionality, subject matter targeted to adults, subject matter targeted to children, subject matter targeted to other age groups, subject matter targeted to different ethnicities, subject matter targeted to different nationalities, subject matter targeted to different cultures, historical subject matter, present time subject matter, futuristic subject matter, subject matter having a certain degree of realism, subject matter including entities with varying degree of relationship closeness, subject matter including celebrities, subject matter including non-celebrities, etc.), and/or other subject matter features, where subject matter aspects can be assessed qualitatively (e.g., categorically) and/or quantitatively (e.g., with scoring). Features of image content can additionally or alternatively be associated with one or more of: size, quality, resolution, type of lens used to capture image content, distortion features, and any other suitable technical feature.

Features of audio content can include subject matter features (e.g., subject matter having a degree of conflict, subject matter having a degree of conflict resolution, subject matter having a degree of love-associated content, subject matter having a degree of adventure-associated content, subject matter having a degree of positive emotionality, subject matter having a degree of negative emotionality, subject matter targeted to adults, subject matter targeted to children, subject matter targeted to other age groups, subject matter targeted to different ethnicities, subject matter targeted to different nationalities, subject matter targeted to different cultures, subject matter having a certain degree of realism, subject matter generated using celebrity voices, subject matter generated using non-celebrity voices, etc.), and/or other subject matter features, where subject matter aspects can be assessed qualitatively (e.g., categorically) and/or quantitatively (e.g., with scoring). Features of audio content can additionally or alternatively be associated with one or more of: duration (of entire audio file, of other subportions of audio), quality, format, verse features, pre-chorus features, chorus features, bridge features, climax features, script features, melody features, beat/meter features, dynamics, harmony features, pitch features, texture features, distortion features, and any other suitable technical feature.

Features of text content (e.g. written content) can include subject matter features (e.g., subject matter having a degree of conflict, subject matter having a degree of conflict resolution, subject matter having a degree of love-associated content, subject matter having a degree of adventure-associated content, subject matter having a degree of positive emotionality, subject matter having a degree of negative emotionality, subject matter targeted to adults, subject matter targeted to children, subject matter targeted to other age groups, subject matter targeted to different ethnicities, subject matter targeted to different nationalities, subject matter targeted to different cultures, subject matter having a certain degree of realism, etc.), storyline aspects, aspects of vernacular used, conversational aspects, language aspects, usability aspects (size, color, font, etc.) and/or other subject matter features, where subject matter aspects can be assessed qualitatively (e.g., categorically) and/or quantitatively (e.g., with scoring).

In embodiments, features of video games and/or game play aspects can include any one or more of: any video features discussed above, any image features discussed above, any audio features discussed above, character personality features, character appearance features, character motion features (e.g., number of movements, rate of movements, etc.), object motion features (e.g., number of movements, rate of movements, etc.), object behavior features, object-character interaction features, object-object interaction features, gameplay physics features, rendering features, environment appearance, environment realism, game play difficulty, power-up features (e.g., boost features, special ability features), duration features, scoring features, story aspect features, and/or any other suitable features.

In embodiments, digital content is provided through one or more devices having one or more of: displays (i.e., for video content output, for image content output), audio output elements, haptic feedback elements, and/or other electronics, embodiments of which are described above. In variations as described above, digital content can be provided through an HMD or other output device; however, digital content can be provided in an alternative manner in other variations. Digital content can be provided contemporaneously with time windows corresponding to collection of neural signal data, or can alternatively be provided in relation to other time windows, as described in Section 2.2 below.

Variations of the method 200 can alternatively omit provision of content to the user, and can instead develop, refine, and implement synthetic brain models without direct provision of the digital content to the users. For instance, a third party entity may provide content directly to users.

### 2.2 Method—Neural Signal Aspects

In relation to receiving 210 a neural signal dataset from a BCI coupled to the user, the BCI collects a neural signal stream and transmits signal aspects to the hardware platform for processing by the computing subsystem, as described above. The components of the system (e.g., the BCI, the hardware platform, the computing subsystem) can thus include detection architecture that allows the system to detect a neural signal stream from the BCI, as the user interacts with or otherwise consumes the digital content. The detection architecture includes structures with operation modes for determining neurological activity (e.g., in relation to spectral content, in relation to neural oscillations, in relation to evoked potentials, in relation to event-related potentials, in relation to different frequency bands of activity, in relation to combinations of activity, etc.), from different electrode channels associated with different brain regions of the user, in order to determine activity states in different regions associated with different brain states.

In embodiments, the different brain states analyzed can include one or more of: an emotional state (e.g., enjoyment, disengagement, interest, boredom, stress, calm, happy, angry, sad, confused, surprised, etc.), an alertness state (e.g., a sleep state, alertness level), a state of focus (e.g., focused, distracted, etc.), a mental health state (e.g., a state of anxiety, a state of depression, a state characterized in a manual of mental health conditions, etc.), a neurological health state (e.g. seizure, migraine, stroke, dementia, etc.), a state of sobriety, a state of overt/covert attention, a state of reaction to sensory stimuli, a state of spatial orientation, a state of cognitive load (e.g. of being overloaded), a state of flow, a state of entrancement, a state of imagery (e.g. of motor action, of visual scenes, of sounds, of procedures, etc.), a memory function state (e.g. encoding effectively, forgetting, etc), and/or any other suitable brain activity state.

The system can collect and process neural signal data contemporaneously with time windows corresponding to provision of digital content, such that neural signal data is collected as the user interacts with a digital content experience provided by the digital content. As such, neural signal data can be collected simultaneously with content provision to users. Neural signal data can alternatively be collected with a suitable temporal offset in relation to content provision to users. However, neural signal data can alternatively be provided in relation to other time windows associated with content provision. Neural signal data can be collected at any suitable rate that provides proper resolution for extraction and classification of neural signal features associated with empathic and/or behavioral responses of users to provided content.

### 2.3 Method—Feature Processing and Classification for Synthetic Brain Model Development and Training

As shown in FIG. 2, the computing subsystem processes 220 the neural signal dataset and a set of features of the digital content experience with a set of classification operations. In embodiments, the classification operations can be constructed with a first set of operations applied to externally-derived data/features (e.g., data/features derived from content experienced by the user(s), data/features derived from signals from the environment(s) of the user(s), etc.) and a second set of operations applied to internally-derived data/features (e.g., signals derived from the brain(s) of the user(s), signals derived from other biometric signals).

In variations, feature extraction methods can be used to process neural signals to extract brain activity-derived features for processing with classification operations, where brain activity-derived features can derived from one or more of: event-related potential data (e.g., voltage-over-time values, etc.); resting state/spontaneous activity data (e.g., voltage-over-time values, etc.); responses to different events (e.g., differences between event-related potentials acquired in response to different events in type, timing, category, etc.); subdivided (e.g., stochastically, systematically, etc.) time-domain signals into numerous atomic units that represent features on different time scales (e.g., subseconds, seconds, minutes, hours, days, months, years, super years, etc.); spectrum aspects (e.g., strike length above and below mean, minima, maxima, positive peaks, negative peaks, time points associated with polarity or other peaks, amplitudes of peaks, arithmetic relationships computed on peaks, etc.); similarity measures (e.g., cosine similarity, various kernels, etc.) between a defined “max-min spectrum” and a pre-computed template; similarity measures between various equivalent atomic units of the raw time domain signals; similarity measures applied to combinations of features; measures derived from clustering of features (e.g., soft/fuzzy clustering, hard clustering); filtered signals (e.g., signals filtered adaptively based on second-order parameters, signals filtered into discrete frequency bands, with computation of variances/autocorrelations in each band, with cross-correlations/covariances across bands); distance metrics (e.g., Riemannian distances) between select matrices; iterative matrix manipulation-derived features (e.g., using Riemannian geometry); entropy measurements within features, between features, and between different user's entire feature sets; subspace analyses (e.g., stationary subspace analyses that demix a signal into stationary and non-stationary sources); recurrence quantification analyses of various time segments; recurrence plots processed through convolutional neural networks that extract additional features (e.g., shapes, edges, corners, textures); principal component analysis-derived features; independent component analysis-derived features; factor analysis-derived features; empirical mode decomposition-derived features; principal geodesic analysis-derived features; sequence probability-derived features (e.g., through hidden Markov models, through application of a Viterbi algorithm, through comparison of probable paths to templates, etc.); single-channel duplication-derived features (e.g., with or without imposition of different distortions to duplicates); invariant features, variable features, and any other suitable brain activity-derived feature. Informative features and their defining weights, for each property of an experience that the synthetic brain models, can be encoded into unique tables that are precomputed in a manner that enables rapid access by future programs and procedures, for example for use in real-time or where efficient computing is needed.

The computing subsystem also applies suitable feature extraction methods for extracting features of digital content provided to users and/or environmental signals, prior to use of such features for training corresponding synthetic brain models.

In variations, multi-way statistical mapping can be implemented at a first level between patterns of features across different modalities to produce new features, and multimodal inference can occur at a level above, based on patterns of classifier outputs from the first level to producing an additional set of features. At each level of feature generation/extraction, synthetic brain models can be trained on inputs that feed into one or more networks (e.g., artificial neural networks, natural neural networks, and/or other deep learning architectures/learning systems), as described. Metaheuristic algorithms can then be applied to the outputs to generate even more precise and reliable models for specific sensorimotor, cognitive, affective, or other states that the empathic system is seeking to model. Models can be generated and/or refined further in any other suitable manner.

To generate features, fused data derived from one or more of user brain activity, and data from other sources (e.g., non-brain-derived sources, auxiliary biometric signals, auxiliary data, etc.) can be processed additionally or supplemental to the nonlinear learning system, by filtering adaptively based on second-order parameters, separating signals into discrete frequency bands, with computation of variances/autocorrelations in each band, with cross correlations and covariances across bands, and inclusion of real and imaginary parts of Fourier transform coefficients in various distance metrics (e.g., Riemannian distances). Mapping between select matrices can be performed using iterative matrix manipulation-derived features (e.g., using Riemannian geometry); entropy measurements within features, between features, and between different user's entire feature sets including sampling at different timescales, and spatially referencing the signal at different locations, real or derived. Inter- and intra-user correlations and other statistically defined relationships, between feature sets, and classifier characteristics, may be used further to transfer models learned across applications using collaborative filtering and other techniques.

### 2.3.1 Method—Synthetic Brain Model Training and Refinement

As shown in FIG. 2, the computing subsystem trains 230 a synthetic brain model with outputs of the set of classification operations and a response dataset characterizing actual responses of the user to the digital content experience, where data acquired in prior method steps and/or through other means is split into a training subportion and a test subportion, in order to improve accuracy of predicted responses using the synthetic brain model. In particular, the synthetic brain model includes architecture for returning outputs associated with predicted user responses to digital content experiences, and is trained as described.

In relation to classification operations in the context of machine learning and training of synthetic brain models, the computing subsystem can implement one or more of the following approaches (for either or both of classification of externally-derived data streams and internally-derived data streams): supervised learning (e.g., using logistic regression, using back propagation neural networks), unsupervised learning (e.g., using an a priori algorithm, using k-means clustering), semi-supervised learning, reinforcement learning (e.g., using a Q-learning algorithm, using temporal difference learning), and any other suitable learning style. Furthermore, the classification and machine learning approaches can implement any one or more of: random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a regression algorithm (e.g., ordinary least squares, logistic regression, stepwise regression, multivariate adaptive regression splines, locally estimated scatterplot smoothing, etc.), a decision tree learning method (e.g., classification and regression tree, iterative dichotomiser 3, C4.5, chi-squared automatic interaction detection, decision stump, a regression method, an instance-based method (e.g., k-nearest neighbor, learning vector quantization, self-organizing map, etc.), a regularization method (e.g., ridge regression, least absolute shrinkage and selection operator, elastic net, etc.), a dimensionality reduction method (e.g., principal component analysis, partial lest squares regression, Laplacian eigenmapping, isomapping, wavelet thresholding, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of algorithm.

Furthermore, metaheuristic and/or non-metaheuristic approaches can also be implemented by the computing subsystem for classification and/or feature-based training approaches. In variations, metaheuristic algorithms applied for classification and development of synthetic brain models can include one or more of: a local search strategy, a global search strategy, a single-solution approach, a population-based approach, a hybridization algorithm approach, a memetic algorithm approach, a parallel metaheuristic approach, a nature-inspired metaheuristic approach, and any other suitable approach. In a specific example, a metaheuristic algorithm applied to internally and/or externally-derived signals can comprise a genetic algorithm (e.g., a genetic adaptive algorithm); however, any other suitable approach can be used. Additionally or alternatively, non-metaheuristic algorithms (e.g., optimization algorithms, iterative methods), can be used.

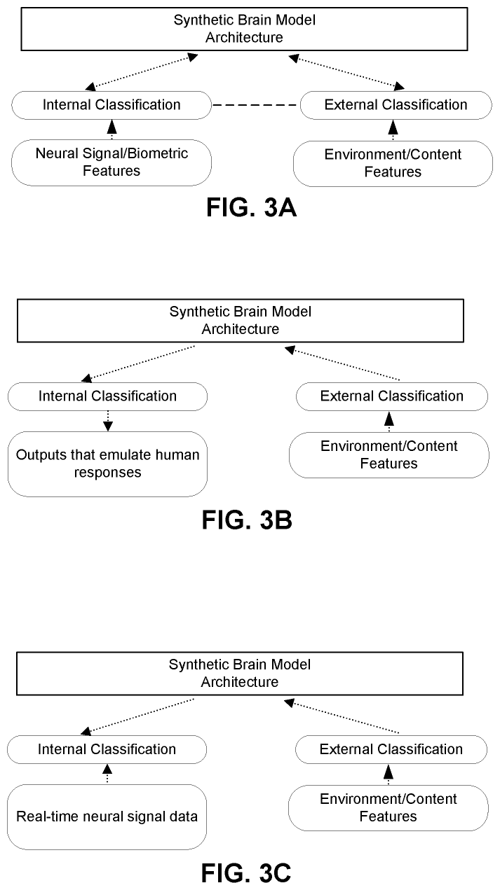

FIG. 3A depicts an embodiment of architecture for classification of externally-derived features and internally-derived features, outputs of which are used to develop, train, and/or otherwise refine synthetic brain models for generating predicted user responses to unevaluated content. In more detail with respect to FIG. 3A, various couplings of two distinct classification systems with corresponding classification operations are configured to capture a range of predicted experiences (e.g., associated with content provision) and responses (e.g., empathic responses of users, behavioral responses of users). In one example, internal classification operations apply a custom-designed artificial neural network (ANN) trained on spatiotemporal features of brain activity, in combination with external classification operations applying a long short-term memory (LSTM) network trained on digital content including video clips, in order to develop a synthetic brain model for predicting responses (e.g., with respect to surprise, with respect to other empathic responses, with respect to perception of content, etc.) to experiences associated with the video clips. In a variation, internal classification operations applying a custom-designed ANN trained on spatiotemporal features of brain activity may alternatively be combined with external classification operations applying Markov models of language to capture experiences related to suspense. In another variation, internal classification operations applying a custom-designed ANN trained on spatiotemporal features of brain activity may alternatively be combined with external classification operations applying Bayesian models of motor execution to capture observation based learning states, or other statistical approaches for clustering and differentiating non-brain data.

Feature layers of the ANNs and other models associated with externally-derived input data (e.g., non-brain or biometric associated data) can be combined with the internally-derived input data to augment the available training data set, and train multinomial models where features are features of different signal types (e.g., brain/biometric vs. content/environment) and outputs of the ultimately trained synthetic brain models are sets of predicted user responses (e.g., in terms of actual experiences, empathic reactions, and/or behavioral responses) labeled either through unsupervised methods or by various forms of self-report, where self-reported inputs are described in more detail below. Transfer learning approaches (e.g. FEDA) and autoencoders can further be used to optimize use of discriminatory information from one data type to enrich another. Co-dependence of variables in the externally-derived factors and in the internally-derived factors may be computed using one or more of: various statistical techniques, k-nearest neighbor measures, fuzzy clustering, and other methods to further augment data for classification.

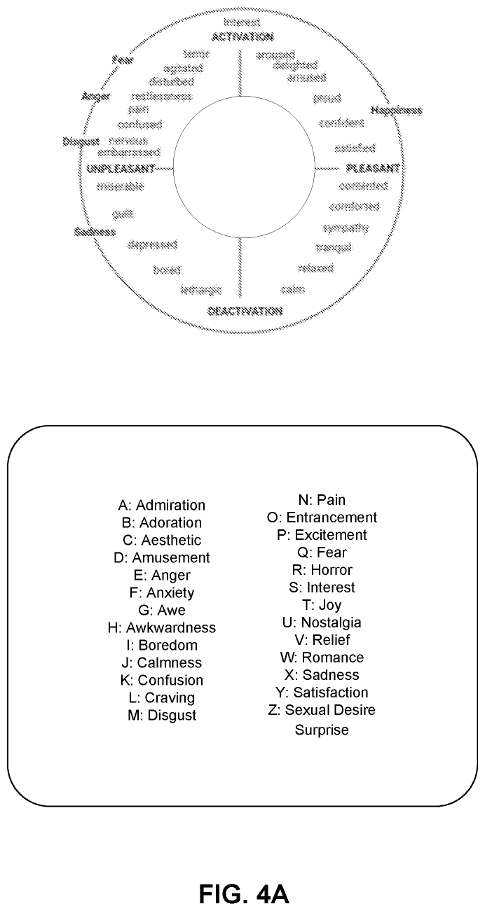

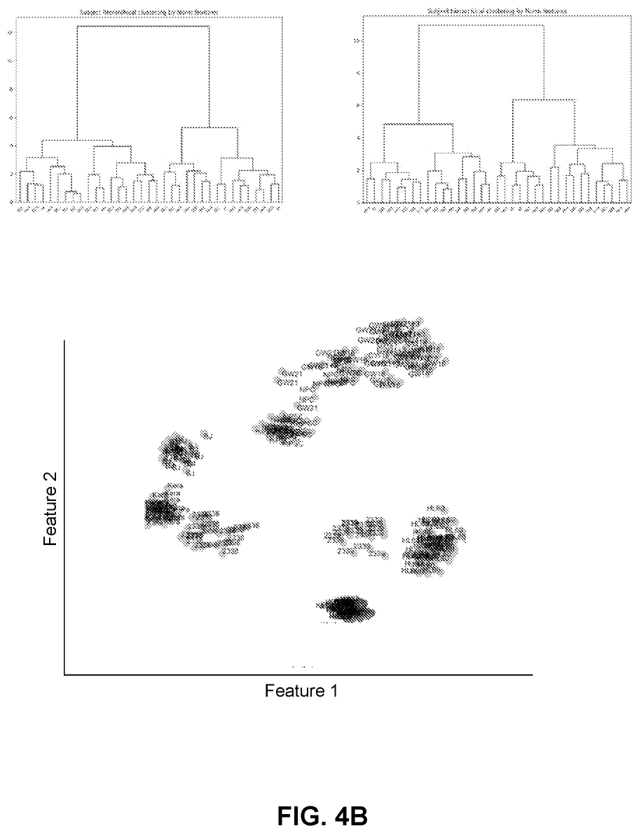

In relation to outputs, the synthetic brain models can be trained to process inputs and return outputs associated with predicted empathic user responses and/or predicted behavioral user responses. In variations, predicted empathic user responses can be categorized according to one or more of: identity, stress, flow, relaxation, joy, sadness, pleasure, discomfort, awe, triumph, excitement, amusement, satisfaction, admiration, aesthetic appeal, boredom, nostalgia, fear, horror, interest, disappointment, anxiety, surprise, sympathy, pride, entrancement, adoration, envy, and other empathic categories. Empathic outputs can further have a score (e.g., in terms of percentages, in terms of a defined metric, in terms of rankings, etc.), or can additionally or alternatively be defined in a binary manner (e.g., this song produced sadness, this song did not produce sadness). Embodiments of various features from models of human emotion are depicted in FIG. 4A, where FIG. 4A (top) depicts a circumplex model (i.e. PAD model) and FIG. 4A (bottom) depicts a second model that maps human emotion to a 27-dimensional space connected at times by smooth gradients, that can be used to develop synthetic brain models.

In variations, predicted behavioral responses can be associated with desired or anticipated actions by users. For instance, predicted behavioral responses in relation to media can include one or more of: addition of content to library or playlist, deletion of content from library or playlist, purchase of content, intent to purchase, likelihood of sharing content, deletion of content, saving of content, addition of content to a wish list, stopping of content prior to completion (e.g., midway through watching a movie, midway through listening to a song), sharing of content with other entities (e.g., through a social platform), and other behavioral responses. Predicted behavioral responses can be determined based upon thresholding analyses, where, the likelihood of the predicted behavior increases when values of a given neural signal feature surpass a threshold condition.

Outputs can further be generated globally for each digital content file. Outputs can additionally or alternatively be generated locally for each segment of a digital content file (e.g., audio clip, movie, etc.). As such, outputs associated with anticipated behavioral responses can be generated for each scene of a movie clip, or each portion of a song (e.g., intro, verse, chorus, bridge, etc.).

As such, in one example, a trained synthetic brain model can return outputs associated with an input music clip, where for each subportion of the music clip a predicted empathic response, with an indication of percent likelihood, is provided. In a specific example, the synthetic brain model can return a prediction that the chorus of a song, which includes lyrics describing an angry breakup scenario, will produce 82% understanding, 71% stress, 57% anger, and 0% relaxation in listeners. However, variations of the specific example can be configured in another manner.

2.3.2 Demographic-Specific Analyses with Processing of Aggregate Data

As shown in FIG. 2, the system can further be configured to refine 235 the synthetic brain model with an aggregate dataset including neural signal data from a population of users, thereby enabling the synthetic brain model to output demographic-related analytics in relation to digital content being evaluated.

In particular, the population of users can include demographics associated with one or more of: gender (e.g., male, female, non-binary), sexual orientation, ethnicity, nationality, age, marital status, household demographics (e.g., sibling relationships, parent features, pets, etc.), geographic location (e.g., places living, placed lived, places traveled to, etc.), health statuses, medical history of individuals/family of individuals, socioeconomic status (e.g., income level), intelligence (e.g., measured by IQ), dietary aspects, level of physical activity, drug use, alcohol use, body mass index-related features, profession, level of education achieved, places of education received, political leanings, criminal history, personality type, history adopting new technologies, and any other suitable demographic features.

Features of the population of users being analyzed can further include social network aspects (e.g., extracted from social network accounts of the users, for instance, through API access, etc.), entertainment preferences (e.g., with respect to genres of video content consumed, with respect to lengths of video content consumed, with respect to styles of audio content consumed, with respect to lengths of audio content consumed, with respect to genres of written media consumed, with respect to formats of media consumed, with respect to genres of gameplay preferred, etc.), and other features.

As such, the computing subsystem can process aggregate data from a large and diverse population of users to refine and expand capabilities of the synthetic brain models, where input data (e.g., related to digital content being analyzed) can be processed with a “demographic-specific” variation of the synthetic brain model to produce desired outputs relevant to selected demographics. In variations, a refined synthetic brain model can be adapted to output predicted responses for particular demographics (e.g., women living in the U.S., children between the ages of 9 and 12, Israeli individuals, college students, etc.).

Alternatively, processing of the aggregate dataset from the population of users can be used by the computing subsystem to identify and/or generate new clusters of individuals, based on identified and/or predicted responses to evaluated digital content. For instance, certain previously unidentified groupings of users can be identified from clusters of similar responses (e.g., through a similarity analysis) to content evaluated using the synthetic brain model. In an example, a cluster of users responding to a portion of an audio clip conveying positive emotions, with sadness, for instance, could be used to identify a new demographic. In still related examples, the computing system can be configured for diagnostic purposes (e.g., in relation to health statuses, in relation to mental health statuses, in relation to undiagnosed health conditions, etc.) based upon such identification of new groups. FIG. 4B depicts example graphs depicting hierarchical clustering of subjects by features determined from neural signals corresponding to user actions (top left) and from neural signals corresponding to user responses to experienced digital content (top right). FIG. 4B (bottom) depicts clustering of subjects by features across multiple dimensions, in order to identify “new” demographics.

2.3.3 Example—Feature Extraction and Classification Approach for Emotional Response Modeling with a Synthetic Brain Model for Arousal and Valence

In an example embodiment, the computing subsystem, in coordination with signal generation by BCI units, processed neural signal data from users as users experienced different digital content (e.g., video content, music content, etc) segments of 60 seconds in duration while several sensors of the BCI and other biometric monitoring devices simultaneously measured their physiological status. In the example, users also completed a subjective questionnaire to provide self-reported data for training the synthetic brain model with respect to outputs associated with responses to the digital content. In more detail, each recorded segment of paired content with neural and other physiological signals was then labeled with a continuous value for valence and arousal between 1 and 9, obtained by the subjective questionnaire.

In the example, a feature extraction procedure was designed specifically for extraction of task-relevant features based on a priori understanding of neural underpinnings of emotions. Features include the log power spectral density in each channel of neural signal data, where the computing system applied a logarithmic transformation to make the features normally distributed. The computing subsystem also implemented a second feature type including a difference in log power spectral density between pairs of corresponding neural signals acquired from both brain hemispheres. In variations of the example, the computing subsystem also extracted features from eye tracking data, heart rate data, and heart rate variability data, as well as other physiological signals, to improve the discriminative power of the classifiers.

In the example, the features were calculated separately for each user and each content segment per user, in a window size determined based on digital content type. For video, for example, feature values for each consecutive one second time window were calculated by the computing subsystem in order to augment the data set, thereby creating more samples for the system to learn relation between samples and the corresponding labels for valence and arousal. In applying the classification and training operations, the computing subsystem determined level of valence and arousal by aggregating classifier outputs on each consecutive ‘one second’ time frame. This aggregation approach significantly improved the accuracy of the predictions.

In this example, two Random Forest classifiers were implemented by the computing subsystem for each new user, where one Random Forest classifier was used for the detection of valence and one Random Forest classifier was used for the detection of arousal. Both classifiers processed only neural signal and task-specific extracted features as an input and returned a value in the range [0, 1] as output, indicating the level of valence and, similarly, the level of arousal experienced by the user. State-of-the-art generalized and transfer learning models were attempted and not found to obtain better results, due to the high variability between subjects.

In the example, the continuous labels, indicating the level of valence and arousal in each trial, in some instances were transformed to dichotomous labels [0,1] making use of a threshold value 5 (in the middle of the [1, 9] interval). This label indicated respectively negative/positive valence or low/high arousal and simplify the problem to a two-class classification problem to reduce the subjective nature of the labels. As such, the example of the method was able to classify responses to provided content, with training subsets of data.

In variations of the example, the computing subsystem was configured to implement one or more of: logistic regression (e.g., with and without label-augmented data), linear discriminant analysis, support vector machines (e.g., with an RBF kernel, with a linear kernel), k-nearest neighbor, random forest approaches (e.g., including heart rate and heart rate variability features), and various transfer functions to generate predicted valence and arousal responses.

### 2.4 Method—Synthetic Brain Model Outputs and Applications

As shown in FIG. 2, with sufficient training and refinement of the synthetic brain models, the computing subsystem processes inputs (e.g., digital content-associated inputs, other “requests”) to return 240 a set of empathic and behavioral outputs associated with predicted user responses to an unevaluated digital content experience. Outputs can be returned as described above in relation to predicted empathic and/or behavioral responses of target users. FIG. 3B depicts an embodiment of implementation of the synthetic brain model trained as described in relation to FIG. 3A. Once the synthetic brain model is trained, input vectors including elements associated with features of video, shows, music, shopping experiences, text, games, and other content can be fed directly to the synthetic brain model, which then processes features of the content and outputs classifications that emulate reactions and feelings of one or more users (e.g., target users). In this way, the empathic computing system can be used as an on-demand tool to modulate content to better serve human users, as well as development test tool to evaluate content, and emulate large focus groups and other screenings of different content, to different groups, etc. FIG. 3C depicts an embodiment of real-time analysis of content data, using the synthetic brain model trained as described in relation to FIG. 3A. According to FIG. 3C, the synthetic brain may also be used in real-time, to analyze content data and give a priori weights to internal classifications, such that the context can inform the analysis. Contribution of the learned experiential models to classification of brain state leads to higher resolution in analytical abilities of the synthetic brain models.

### 2.4.1 Example Outputs Corresponding to Synthetic Brain Model Processing of Music and Video Content

In example implementations of a synthetic brain model, a selection of songs was processed with the synthetic brain model and a subportion of songs were predicted to produce more relaxation compared to other contemporary releases. In particular, one song was predicted (and subsequently verified) to produce the most happiness in listeners. Additional outputs included the following: men were predicted to enjoy one song more than women at all age groups tested, men were predicted to experience more happiness in the first half of the song than the last half of the song. Surfers were predicted to find a song less boring than the rest of the demographics tested. Yoga listeners were predicted to be ambivalent to two of three songs.

Additionally, the synthetic brain model output predicted “hit” scores based upon combine multiple metrics related to predicted empathic and/or behavioral responses, in order to determine the anticipated success of song. In testing accuracy of “hit” score predictions, the computing system generated correlations between “hit” score predictions and with data from music streaming and purchasing platforms (e.g., in relation to rankings, in relation to number of downloads, in relation to number of purchases, in relation to number of additions to playlists, in relation to number of repeated listening events, etc.).

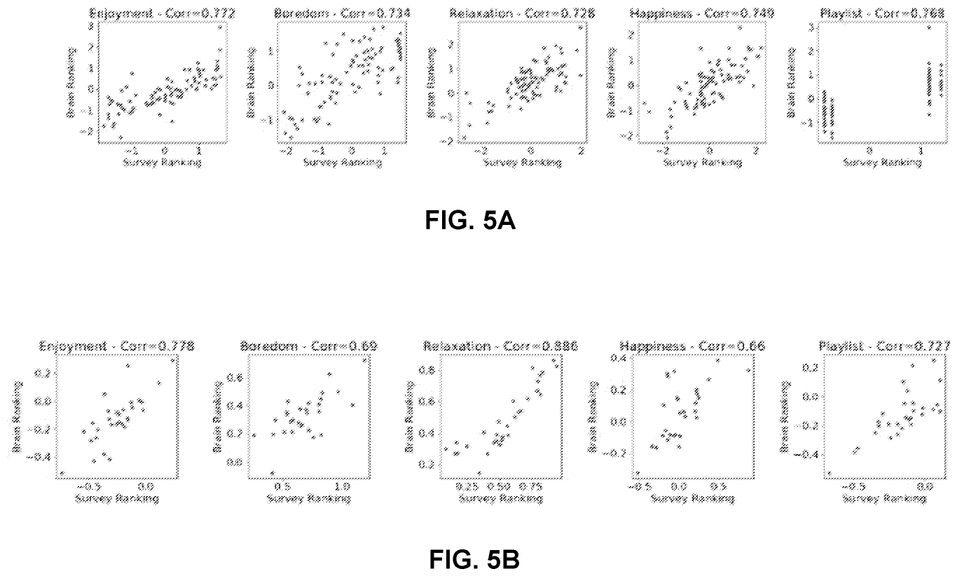

FIG. 5 depicts a series of charts correlating example outputs of a synthetic brain model to self-reported responses to consuming of provided content by users. In more detail, the synthetic brain model received an input vector associated with music clip features, and generated outputs associated with predicted enjoyment, boredom, relaxation, happiness, and behavior (e.g., with respect to addition of the music to a playlist), and the computing subsystem correlated outputs of the synthetic brain model with self-reported responses by the users being evaluated, thereby indicating accuracy of predictions. As such, the synthetic brain model was capable of returning analytics in relation to real-time experiences of users as the users experienced content, in addition to reflective considerations of users after the content was experienced. In particular, self-reported responses are only able to provide reflective feedback.

In an application related to FIG. 5A, FIG. 5B depicts example model outputs generating predictions of user responses, according to operations described above, where response predictions were generated for different demographics (e.g., pop fans, genders, surfers, ages, yoga-ists, locations, etc.).

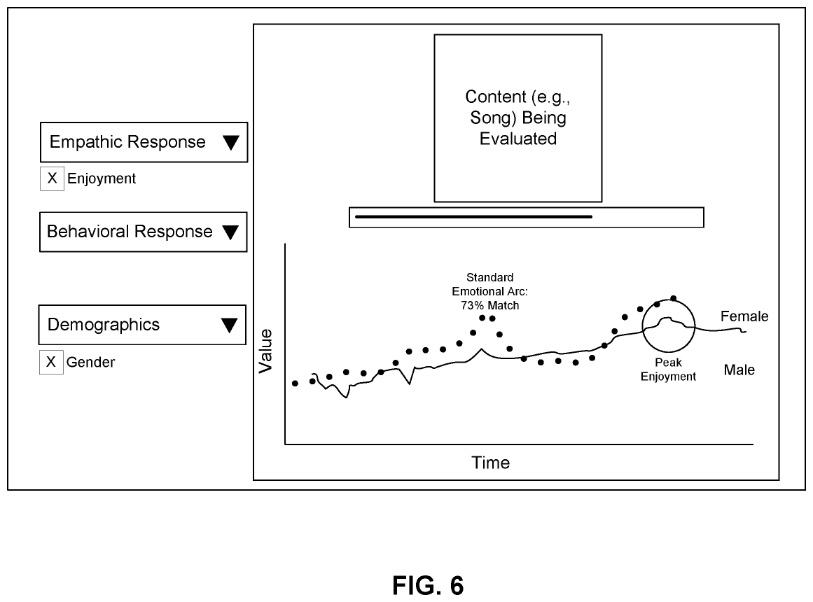

FIG. 6 depicts example output metrics related to empathic responses (e.g., relaxation, anxiety, enjoyment, boredom, etc.) and other responses across each segment of and through an entire duration of evaluated song, for different demographics (e.g., male, female, American, Israeli, other nationality, etc.). For each category of empathic output (e.g., boredom prediction), the synthetic brain model was configured to return indications of peak events (e.g., peak boredom at time point 2:32, peak enjoyment at time point 0:20, etc.). The synthetic brain model was also configured to return indications of emotional arcs (in one or more empathic response categories), across content (e.g., song), and to generate comparisons between emotional arc characteristics between different evaluated songs. In related examples, aspects of emotional arcs, were correlated with success measures, and mapped to technical features of songs (e.g., melody features, beat features, lyrical features, etc. as described above. As such, the synthetic brain model was used to provide analytics of features correlated with content success, in relation to emotional arc characteristics of songs. Such outputs can be adapted to other media formats (e.g., video content, text content, gaming content, shopping experiences, etc.). Furthermore, variations of the methods can produce outputs with error bars or error ranges, in order to provide measures of confidence in predictions or other determined outputs.

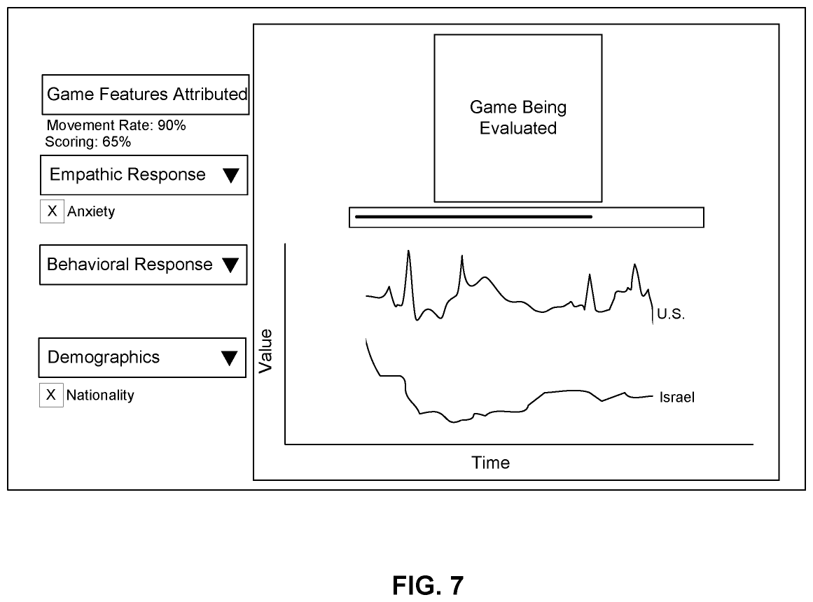

FIG. 7 depicts output metrics produced by a synthetic brain model, where outputs are related to empathic responses (e.g., relaxation, anxiety, enjoyment, boredom, engagement, difficulty, etc.) in relation to different game play factors of a video game experience.

In relation to a block packing game, input features of video game content included one or more of: round duration, number of block movements (e.g., shifts, rotations), rates of movements, numbers of speed boosting events, duration of speed boosting events, set times, numbers of winning events, recovery difficulty, and score. In relation to a block breaking game, input features of video game content included one or more of: round duration, number of paddle movements, rate of paddle movements, paddle position, ball position, number of paddle hits, number of block hits, and score. Game play features for other games can include other game elements, level difficulty aspects, character skins, environment skins, and other features described above.

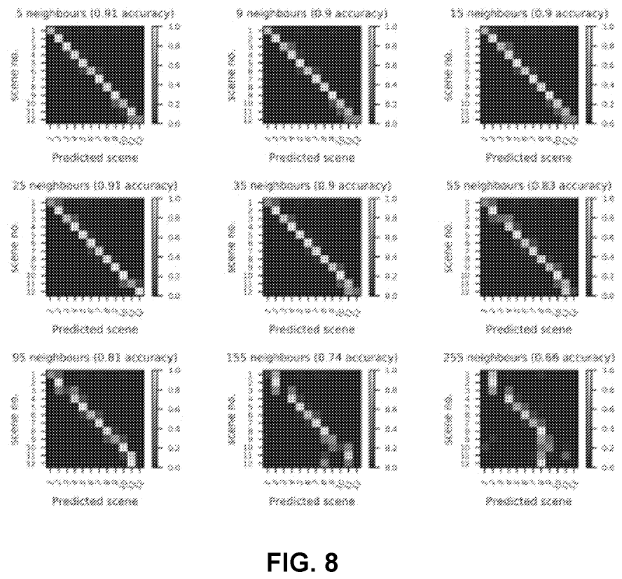

FIG. 8 depicts examples graphics corresponding to outputs of synthetic brain models used to process input neural signal data, in order to produce output predictions of portions of digital content being consumed (based on evaluation of neural signal data alone). In generating outputs associated with FIG. 8, input neural signals derived from brain activity (e.g., brain-recorded data epochs) of users was used by the computing subsystem, with a cross-correlation analysis, to predict, from brain activity data alone, what video content users were watching. In more detail, the computing subsystem implemented data processing and feature extraction operations (as described above) to improve classification accuracy in relation to outputs capturing predictions of what users were consuming (e.g., a kissing scene, a scene of a travelling couple, a parachuting scene, a scene involving disgusted expressions, an angry phonecall scene, a violent scene, a birthing scene, a family scene, etc.). FIG. 8 depicts example plots demonstrating high correlations between predicted content being watched, and actual content being watched by users, where prediction accuracy was affected by brain data epoch size, feature composition (e.g., of neural signal data), number of components being analyzed, signal filtering operations, window sizes, and stride. Variations of the example can be adapted to predictions of other types of media being consumed and/or other experiences of users (e.g., real life experiences as a user is going about his/her daily life), based on analysis of neural signal data alone. Such outputs cannot typically be generated in near real time outside of more involved and costly imaging modalities (e.g., MRI) that are not practical at an industrial scale.

### 2.5 Method—Targeting and Generative Applications of Synthetic Brain Model Outputs

As shown in FIG. 2, the computing subsystem processes outputs of the synthetic brain model and executes 250 an action in response to the set of empathic and behavioral outputs. As such, outputs of the synthetic brain models can be used as new inputs for machines and other systems for producing improvements in the real world.

In embodiments, the actions executed in response to outputs of the synthetic brain model can include one or more of: targeted marketing of evaluated content to population subsets in a strategic manner; actions associated with modulation or generation of digital content, with improvements; actions associated with controlling operation of connected devices; and any other suitable action(s).

### 2.5.1 Strategic Content Targeting

In variations, targeted marketing of evaluated content to population subsets in a strategic manner can include: based on outputs indicating more positive responses from specific demographics to a piece of digital content (e.g., song, movie, TV show, video game, book, article, consumable, item for purchase, etc.), automatically promoting the digital content to the specific demographics (e.g., through social networks, through targeted advertising platforms, through mass mailing, etc.). As such, the method can include executing an action, where the action comprises a targeting action, the targeting action comprising automatic dispersion of digital content derived from the unevaluated digital content experience to a subpopulation of users predicted to respond positively to unevaluated digital content. In variations, the subpopulation of users belong to at least one of: an age group demographic, a gender demographic, a nationality demographic, and a geographic location demographic predicted to respond positively to the unevaluated digital content. In one example, in response to generating outputs indicating that certain age groups respond positively to evaluated content, regardless of geographic location, the computing subsystem can disseminate the evaluated content to the target age groups widely across geographic locations not previously targeted. In another example, in response to generating outputs indicating that demographics from certain geographic locations respond less positively to content, the computing subsystem can automatically generate a plan to avoid targeting of certain geographic locations with the content (e.g., through targeted advertising, etc.). This example can be applied to automatic planning of a tour for a music band, such that they do not waste efforts in less receptive areas and maximize value. As such the synthetic brain model can efficiently and rapidly process digital content features and predict responses to the content in order to guide strategic targeting efforts. In another example, the computing subsystem can apply outputs of the synthetic brain models to selection algorithms for subscription-based content provision services (e.g., providing digital content to be watched, listened to, read, etc.), in order to design more engaging queues of content for different demographics. Outputs can be used for strategic targeting of content, based on synthetic brain outputs, in another manner.

### 2.5.2 Content Modulation and Generation

In variations, the computing subsystem can additionally or alternatively apply outputs of synthetic brain model to perform actions associated with modulation or generation of digital content. In particular, the computing subsystem can apply desired or undesired response patterns (e.g., in terms of negative responses, in terms of emotional arc aspects, etc.) associated with outputs of the synthetic brain models to perform Boolean operations on content (e.g., in relation to cutting portions of digital content, in relation to adding portions of digital content, in relation to affecting play rates of digital content, in relation to affecting speeds of digital content, in relation to adjusting intensities of portions of digital content, in relation to generating repeats of content with or without modification, etc.). Boolean operations can be applied to content of any format (e.g., video, audio, games, text, haptic, etc.) being evaluated. Furthermore, Boolean operations can be automatically applied, or can alternatively be applied with generation of instructions for another entity or computing subsystem to apply (e.g., in a semi-autonomous or manual manner).

In an example, outputs indicating that a movie has scenes that produced undesired disturbed empathic responses can be used to automatically trim or eliminate such portions of the movie clip in a new file. In another example, outputs indicating that a song produces unexpected positive responses between time points 2:12 and 2:31 can be used to duplicate technical features of the song file present between time points 2:12 and 2:31 in another portion of the song. In another example, outputs indicating that engagement with content decreases at a certain point of the digital content experience can be used to increase impact of features associated with enjoyment prior to the “disengagement period”, in order to reduce likelihood of disengagement.

In another example, outputs indicating that video game character features or rates of movement produce boredom can be used to adjust character appearance features and increase rates of movement, in order to generate improved gameplay aspects. In another example, real or near-real time outputs capturing empathic responses of users can be used to provide live game adaptation (e.g., in both the game features and the surroundings in the user's environment through connected devices controlling audio, light, and other outputs), thereby creating an immersive and deeply engaging experience in real-time. As such, in examples, features of gameplay and environment can be tested in relation to predicted responses of a wide population of users before mass release.

In another example, outputs indicating that voice feature characteristics of a virtual assistant are annoying can be used to modulate the voice features (e.g., in relation to intonation, in relation to language trees, in relation to speed of speech, etc.) to produce a less annoying virtual assistant. Additionally or alternatively, in another example, outputs indicating that timing of assistance from a virtual assistant contributing to reduced engagement (e.g., in relation to responses of users) can be used to adjust timing of assistance such that it produces higher engagement. Generative actions can additionally or alternatively be applied to content of various formats in another manner. In another example, the computing subsystem can apply outputs of synthetic brain models used to process a novel, in order to generate suggestions for storyline feature modulation (e.g., in relation to fantastical elements, in relation to dramatic elements, in relation to character development, in relation to other aspects), in order to improve engagement.

In relation to generative actions, the method can additionally or alternatively include re-evaluation of modulated or generated content, with the synthetic brain models, in order to determine if such modulation or generation produced improved content.

Additionally or alternatively, in relation to generative actions, the method can include A/B testing versions of features being evaluated across different digital content files (e.g., for different movie clips/trailers, for different songs, for different gameplay features, etc.), across selected demographics.

### 2.5.3 Adjusting Operation of Other Systems

In variations, the computing subsystem can additionally or alternatively apply outputs of the synthetic brain models to perform actions associated with controlling operation of connected devices or other platforms. For instance, variations of the system that incorporate BCI units coupled to users can process neural signals and determine, based upon analysis of empathic responses, actions that a user desires to perform. For instance, outputs indicating that a user had a positive response to an online shopping experience and desires to purchase an item can be used to generate instructions for automatically purchasing the item captured in the shopping experience. In variations, the action can additionally or alternatively comprise one or more of: adding the item to a shopping cart, deleting the item from the shopping cart, adding the item to a wish list, etc.

In another example, outputs capturing empathic and behavioral responses of users can be used to generate control instructions for connected devices in environments of users, in order to improve user cognitive states. Such connected devices can include one or more of: audio output devices, light output devices, virtual reality equipment, heat controlling devices, connected appliances (e.g., coffee makers, ovens, etc.), and other devices.

In another example, the computing subsystem can combine analyses of intended behaviors of users determined through neural signal analysis, with outputs of empathic response models, in order to verify and initiate user actions. As such, the computing subsystem can perform checks to see if it made a mistake based on the emotional response of the user's brain following initiation of what the computing subsystem determined to be the intended action of the user.

### 3. Conclusion

The systems and methods described can confer benefits and/or technological improvements, several of which are described below:

The systems and methods can rapidly decode user brain activity states and dynamically generate synthetic brain models for evaluating digital content, with receipt of signals from brain computer interfaces. In particular the system includes architecture for rapidly decoding user states in a manner that can be used to provide digital content to demographics in a desired manner. As such, the systems and methods can improve function of predictive computing platforms, devices for generation of digital content, virtual reality, augmented reality, and/or brain computer interface devices relation to improved content delivery through devices that are subject to limitations in functionality.

The systems and methods can additionally efficiently process and deliver large quantities of data (e.g., neural signal data) by using a streamlined processing pipeline. Such operations can improve computational performance for data in a way that has not been previously achieved, and could never be performed efficiently by a human. Such operations can additionally improve function of a system for delivering digital content to a user, where enhancements to performance of the virtual system provide improved functionality and application features to users of the virtual system.

Furthermore, the systems and methods generate novel training data, synthetic brain models in a way that has not been achieved before, with real-world applications.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described. The computer can be a specialized computer designed for user with a virtual environment.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method for synthetic brain refinement and implementation, the method comprising:
providing a digital content experience to a user;
receiving a neural signal dataset from a brain computer interface coupled to the user, as the user interacts with the digital content experience,
processing the neural signal dataset and a set of features of the digital content experience with a set of classification operations;
training a synthetic brain model with outputs of the set of classification operations and a response dataset characterizing actual responses of the user to the digital content experience, the synthetic brain model comprising architecture for returning outputs associated with predicted user responses to digital content experiences;
refining the synthetic brain model with an aggregate dataset comprising neural signal data from a population of users;
returning a set of empathic and behavioral outputs associated with predicted user responses to an unevaluated digital content experience, upon processing the unevaluated digital content experience with the synthetic brain model; and
executing an action in response to the set of empathic and behavioral outputs.

2. The method of claim 1, wherein the digital content experience comprises one or more of: an audio listening experience, a video watching experience, an image viewing experience, a text reading experience, a shopping experience, and a video gameplay experience provided by way of a digital content file.

3. The method of claim 1, wherein the set of classification operations comprises a first subset of operations applied to externally derived features comprising the set of features of the digital content experience and environmentally-derived signals, and a second subset of operations applied to the neural signal dataset and biometric features.

4. The method of claim 1, wherein features neural signal data are derived from at least one of: event-related potentials, spatiotemporal aspects, spectrum aspects, and distance features across feature matrices.

5. The method of claim 1, wherein the set of empathic and behavioral outputs comprises empathic outputs characterizing one or more of: boredom, joy, flow, anger, stress, sadness, and relaxation experienced by a target audience of the unevaluated digital content experience.

6. The method of claim 1, wherein the set of empathic and behavioral outputs comprises behavioral outputs characterizing one or more of: addition of content to at least one of a library and a playlist, deleting of content from at least one of the library and the playlist, stopping content playback, and a purchasing action by a target audience of the unevaluated digital content experience.

7. The method of claim 1, wherein the population of users comprises users of a set of demographics comprising at least one of: an age group demographic, a gender demographic, a nationality demographic, and a geographic location demographic, and wherein the synthetic brain model is configured to return the set of empathic and behavioral outputs for a selected demographic of the set of demographics.

8. The method of claim 1, wherein the action comprises a generative action applied to a digital content file associated with the unevaluated digital content experience, wherein the generative action comprises a Boolean operation applied to the digital content file.

9. The method of claim 1, wherein the action comprises a targeting action, the targeting action comprising automatic dispersion of digital content derived from the unevaluated digital content experience to a subpopulation of users predicted to respond positively to unevaluated digital content.

10. A method for synthetic brain implementation, the method comprising:
receiving a set of features of an unevaluated digital content experience;
processing the set of features with a synthetic brain model,
wherein the synthetic brain model is trained with outputs of a set of classification operations applied to neural signal data from a population of users, features of digital content experiences, and a response dataset characterizing actual responses of users to digital content experiences;
upon processing the set of features with the synthetic brain model, returning a set of empathic and behavioral outputs associated with predicted user responses to the unevaluated digital content experience; and
executing an action in response to the set of empathic and behavioral outputs.

11. The method of claim 10, wherein the unevaluated digital content experience comprises one or more of: an audio listening experience, a video watching experience, an image viewing experience, a text reading experience, a shopping experience, and a video gameplay experience provided by way of a digital content file, and wherein the set of features comprise subject matter features configured to produce emotional responses in users.

12. The method of claim 10, wherein training the synthetic brain model comprises implementing at least one of: a random forest operation, a long short-term memory operation, an artificial neural network operation, and a metaheuristic operation.

13. The method of claim 10, wherein the set of empathic and behavioral outputs comprises empathic outputs characterizing one or more of: boredom, joy, flow, anger, stress, sadness, and relaxation experienced by a target audience of the unevaluated digital content experience.

14. The method of claim 1, wherein the set of empathic and behavioral outputs comprises behavioral outputs characterizing one or more of: addition of content to at least one of a library and a playlist, deleting of content from at least one of the library and the playlist, stopping content playback, and a purchasing action by a target audience of the unevaluated digital content experience.

15. The method of claim 10, wherein the action comprises a generative action applied to a digital content file associated with the unevaluated digital content experience, wherein the generative action comprises a trimming action applied to portions of the digital content predicted to produce a negative response, based the set of empathic and behavioral outputs.

16. The method of claim 1, wherein the action comprises a targeting action, the targeting action comprising automatic dispersion of digital content derived from the unevaluated digital content experience to a subpopulation of users predicted to respond positively to unevaluated digital content, wherein the subpopulation of users belong to at least one of: an age group demographic, a gender demographic, a nationality demographic, and a geographic location demographic predicted to respond positively to the unevaluated digital content.

17. A method for synthetic brain refinement, the method comprising:
providing a digital content experience to a user;
receiving a neural signal dataset from a brain computer interface coupled to the user, as the user interacts with the digital content experience,
processing the neural signal dataset and a set of features of the digital content experience with a set of classification operations;
training a synthetic brain model with outputs of the set of classification operations and a response dataset characterizing actual responses of the user to the digital content experience, the synthetic brain model comprising architecture for returning outputs associated with predicted user responses to digital content experiences; and
refining the synthetic brain model with an aggregate dataset comprising neural signal data from a population of users.

18. The method of claim 17, wherein the neural signal dataset comprises a set of spatiotemporal brain activity features for training of the synthetic brain model, and wherein the set of features of the digital content experience comprises features configured to produce an emotional response.

19. The method of claim 17, wherein the set of empathic and behavioral outputs comprises empathic outputs characterizing one or more of: boredom, joy, flow, anger, stress, sadness, and relaxation experienced by a target audience of the digital content experience, wherein the empathic outputs are provided for each segment across a duration of the digital content experience.

20. The method of claim 17, wherein the population of users comprises users of a set of demographics comprising at least one of: an age group demographic, a gender demographic, a nationality demographic, and a geographic location demographic, and wherein the synthetic brain model is configured to return the set of empathic and behavioral outputs for a selected demographic of the set of demographics.
